(19) United States
(12) Patent Application Publication
(10) Pub. No.: US 2010/0021994 A1
(43) Pub. Date: Jan. 28, 2010

(54) APPARATUS FOR DETERMINING THE TERMINATION OF FAT DIGESTION AND FAT TISSUE DIGESTION APPARATUS

(75) Inventor: Chisato Karasawa, Saitama (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/527,126

(22) PCT Filed: Feb. 4, 2008
(86) PCT No.: PCT/JP2008/051721

§ 371 Date: Aug. 13, 2009

(30) Foreign Application Priority Data
Feb. 16, 2007 (JP) 2007-036574

(51) Int. Cl. C12M 1/00

(52) U.S. Cl. 435/288.7; 435/287.1

(57) ABSTRACT

To enable to yield a group of fat-derived stem cells of constant quality regardless of the individual difference in fat tissue. It is intended to provide an apparatus for determining the termination of fat digestion (1) for use in a fat tissue digestion apparatus which digests a fat tissue by placing and stirring the fat tissue with an enzyme-containing physiological saline, lactate Ringer's solution, or buffer solution in a container, wherein the apparatus for determining the termination of fat digestion comprises: a boundary-detecting unit (8) which detects the boundary between a fat tissue layer formed in the container by being left still after stirring and a cell suspension layer located below the fat tissue layer, from outside the container; a layer thickness-measuring unit (10) which measures the thickness of the fat tissue layer based on the position of the boundary detected by the boundary-detecting unit (8); a fat volume-calculating unit (12) which calculates the volume of the fat tissue layer by multiplying the thus measured thickness of the fat tissue layer by the cross-sectional area of the container; and a termination-determining unit (13) which determines the termination of digestion based on the thus calculated volume of the fat tissue layer 120

### TECHNICAL FIELD

The present invention relates to an apparatus for determining the termination of fat digestion and a fat tissue digestion apparatus for yielding fat-derived stem cells through digestion of a fat tissue.

### BACKGROUND ART

Conventionally, there has been known a technique for separating fat-derived stem cells contained in fat tissue by collecting a human fat tissue and stirring it with a digestive enzyme and a physiological saline (for example, see Patent Document 1).

In this technique, the digestion goes on with stirring, and the fat tissue is decomposed to thereby separate the fat-derived stem cells. The termination of this digestion is determined by counting the time required for digestion.

Patent Document 1:

PCT International Publication No. WO 2005/012480 Pamphlet

### DISCLOSURE OF INVENTION

However, the fat tissue digestion largely varies depending on the individual difference, which leads to a problem in that a group of fat-derived stem cells of constant quality can not be obtained if the termination of fat tissue digestion is determined according to the elapsed time. In addition, in order to obtain a group of sufficient fat-derived stem cells regardless of the individual difference in fat tissue, the digestion treatment needs to be performed over a long period of time to meet the time for treating a fat tissue which is slow to be digested. Thus, the treating time can not be inconveniently shortened.

The present invention takes the above situation into consideration with an object of providing an apparatus for determining the termination of fat digestion and a fat tissue digestion apparatus which are capable of yielding a group of fat-derived stem cells of constant quality regardless of the individual difference in fat tissue.

In order to achieve the above object, the present invention provides the following solutions.

A first aspect of the present invention is an apparatus for determining the termination of fat digestion for use in a fat tissue digestion apparatus which digests a fat tissue by placing and stirring the fat tissue with an enzyme solution, and any one of a physiological saline, a lactate Ringer's solution, and a buffer solution in a container, wherein the apparatus for determining the termination of fat digestion comprises: a boundary-detecting unit which detects the boundary between a fat tissue layer formed in the container by being left still after stirring and a cell suspension located below the fat tissue layer, from outside the container; a layer thickness-measuring unit which measures the thickness of the fat tissue layer based on the position of the boundary detected by the boundary-detecting unit; a fat volume-calculating unit which calculates the volume of the fat tissue layer by multiplying the thus measured thickness of the fat tissue layer by the cross-sectional area of the container; and a termination-determining unit which determines the termination of digestion based on the thus calculated volume of the fat tissue layer.

According to the first aspect, the boundary between the fat tissue and the cell suspension in the container is detected through the operation of the boundary-detecting unit, the thickness of the fat tissue layer is measured based on the position of the boundary through the operation of the layer thickness-measuring unit, and the volume of the fat tissue layer is calculated based on the thickness of the fat tissue layer and the cross-sectional area of the container through the operation of the fat volume-calculating unit. The fat tissue ends up being a cell suspension as the digestion goes on, because the tissue-connecting collagen is decomposed and thereby microstructures such as cells and extracellular tissue are lysed into a physiological saline. For this reason, the thickness of the fat tissue layer becomes thinner as the digestion goes on. Accordingly, the degree of fat tissue digestion can be accurately determined by calculating the volume of the fat tissue layer, and the termination of digestion can be determined by the operation of the termination-determining unit so that the digestion treatment can be terminated at the time when a predetermined amount of the fat tissue has been digested regardless of the individual difference in fat tissue, to yield a group of fat-derived stem cells of constant quality in a short time.

In the first aspect, the structure may be such that the container is transparent, and the boundary-detecting unit comprises a camera which is arranged to face a side wall of the container for capturing an image of the fat tissue layer inside the container through the side wall of the container.

By so doing, the boundary between the fat tissue layer and the cell suspension can be readily detected by processing the image of the fat tissue layer fat tissue layer inside the container that has been captured by the camera, and the termination of fat tissue digestion can be readily determined.

In addition, in the first aspect, the structure may also be such that the container is transparent and the boundary-detecting unit comprises a light emitting element and a light receiving element which are opposingly arranged to interpose the container.

By so doing, the boundary between the fat tissue layer and the cell suspension can be readily detected based on the amount of light received by the light receiving element out of the amount of light emitted from the light emitting element, utilizing the phenomenon that the optical absorbance differs between the fat tissue layer and the cell suspension because of their difference in the physical property.

In addition, the above structure may also be such that a plurality of the light emitting elements and the light receiving elements are arranged in a vertical direction of the container.

By so doing, even though the position of the boundary between the fat tissue layer and the cell suspension varies at the time when the termination of fat tissue digestion is determined, due to the difference in the amount of fat tissue, any one of the physiological saline, the lactate Ringer's solution, and the buffer solution placed in the container, the boundary position can be readily detected by any of the plurality of the vertically arranged light emitting elements and light receiving elements.

In addition, the above structure may also comprise a movement mechanism which moves the light emitting element and the light receiving element in the vertical direction of the container.

By so doing, even though the position of the boundary between the fat tissue layer and the cell suspension varies at the time when the termination of fat tissue digestion is determined, due to the difference in the amount of fat tissue, any one of the physiological saline, the lactate Ringer's solution, and the buffer solution placed in the container, the boundary position can be readily detected by vertically moving the light emitting element and the light receiving element through the operation of the movement mechanism.

A second aspect of the present invention is a fat tissue digestion apparatus comprising the fat digestion termination-determination apparatus of any structure mentioned above.

According to the second aspect, the amount of undigested fat tissue remaining in the container can be accurately detected through the operation of the fat digestion termination-determination apparatus, and a group of fat-derived stem cells of constant quality can be obtained.

The present invention demonstrates an effect in which a group of fat-derived stem cells of constant quality can be yielded regardless of the individual difference in fat tissue.

### BRIEF DESCRIPTION OF DRAWINGS

FIG. 1 shows an overall configuration of a fat tissue digestion apparatus according to one embodiment of the present invention.

FIG. 2 is a front view showing a fat digestion termination-determination apparatus to be provided in the fat tissue digestion apparatus of FIG. 1.

FIG. 3 is a block diagram showing the fat digestion termination-determination apparatus of FIG. 2.

FIG. 4 is a perspective view showing a first modified example of the fat digestion termination-determination apparatus of FIG. 2.

FIG. 5 is a perspective view showing a second modified example of the fat digestion termination-determination apparatus of FIG. 2.

FIG. 6 is a perspective view showing a third modified example of the fat digestion termination-determination apparatus of FIG. 2.

FIG. 7 is a perspective view showing a fourth modified example of the fat digestion termination-determination apparatus of FIG. 2.

FIG. 8 is a longitudinal section of a fifth modified example of the fat digestion termination-determination apparatus of FIG. 2.

### EXPLANATION OF REFERENCE SIGNS

A: free lipid layer (fat tissue layer)
B: tissue layer (fat tissue layer)
C: cell suspension layer
D: boundary
1: fat digestion termination-determination apparatus
2: fat tissue digestion apparatus
3: transparent container (container)
8: camera (boundary-detecting unit)
10: layer thickness-measuring unit
12: fat volume-calculating unit
13: termination-determining unit (digestion termination-determining unit)
20: infrared photoelectronic sensor (boundary-detecting unit)
20a: light emitting element
20b: light receiving element
22: lifting mechanism (movement mechanism)
24: capacitive sensor

### BEST MODE FOR CARRYING OUT THE INVENTION

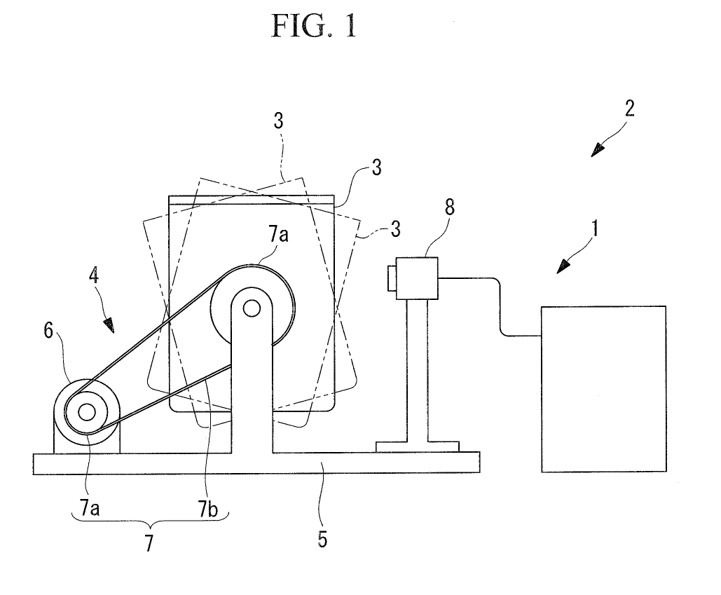

Hereunder is a description of a fat digestion termination-determination apparatus 1 and a fat tissue digestion apparatus 2 according to a first embodiment of the present invention, with reference to FIG. 1 through FIG. 3.

As shown in FIG. 1, the fat tissue digestion apparatus 2 according to this embodiment comprises a transparent container 3, a swing mechanism 4 which swings the transparent container about an approximate horizontal axis, and the fat digestion termination-determination apparatus 1 according to this embodiment. The transparent container 3 is an approximately cylindrical container whose inner cross-sectional area is unchanged in the vertical direction.

A fat tissue collected from a human or another mammal, a protease such as trypsin, and a physiological saline (alternatively a lactate Ringer's solution or a buffer solution) can be placed in the transparent container 3. The placed fat tissue starts float upward in the physiological saline due to the relation of their specific gravities, and forms a fat tissue layer by being left still. The volume of the fat tissue is measured in advance before the fat tissue is placed in the container, and this data is stored in a memory unit that will be described later.

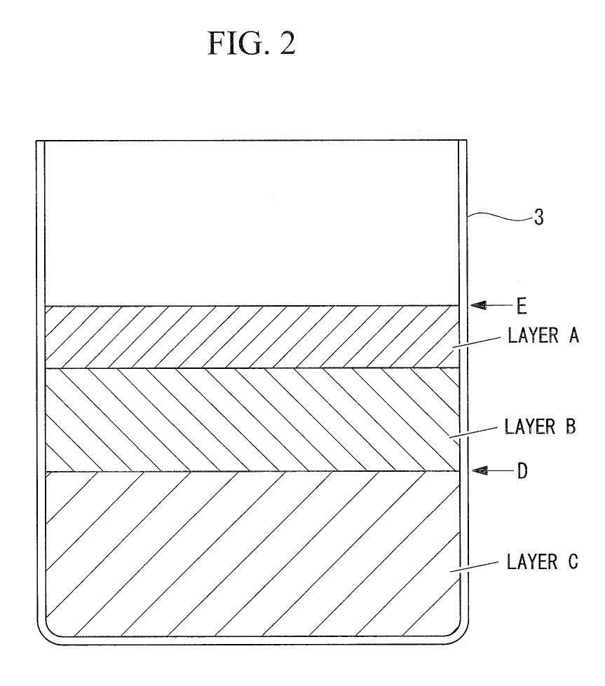

As shown in FIG. 2, the fat tissue placed in the enzyme-containing physiological saline is separated into a free lipid layer (layer A) composed of oil and oil-containing cells, a cell suspension layer (layer C) containing the physiological saline and digested cells, and an undigested tissue layer (layer B) located between these free lipid layer and cell suspension layer and containing both components. Then, with the passage of time, the layer A alters into the layer C through the layer B. In this embodiment, the term “fat tissue layer” means the free lipid layer A and the tissue layer B.

The swing mechanism 4 comprises a base 5 which swingably supports the transparent container 3, a motor 6, and a transmission mechanism 7 which is rotatably driven by the motor 6 to swing the transparent container 3 supported by the base 5. The transmission mechanism 7 comprises, for example, a pulley 7a and a belt 7b.

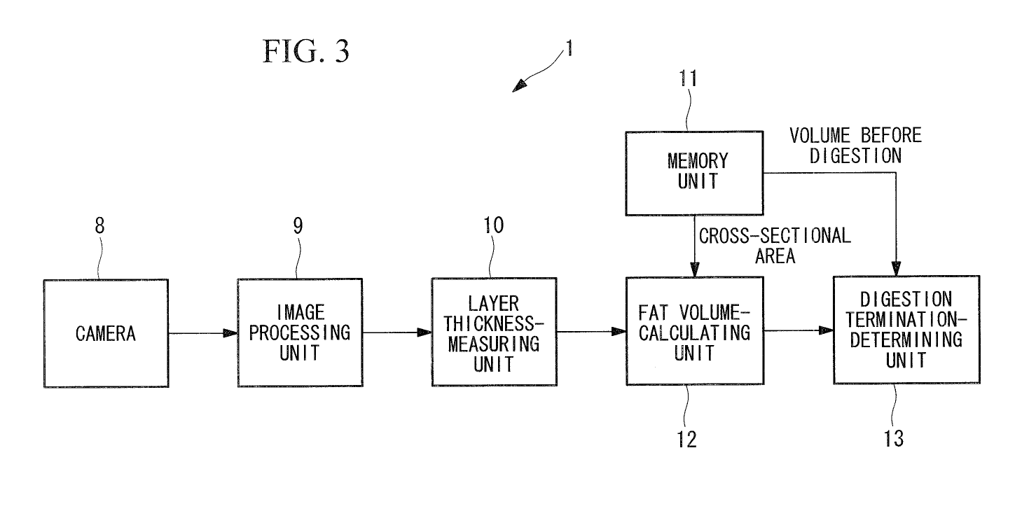

As shown in FIG. 3, the fat digestion termination-determination apparatus 1 comprises a camera 8 arranged to face the outer surface of the side wall of the transparent container 3, an image processing unit (boundary-detecting unit) 9 which processes an image captured by the camera 8 to detect the boundary D between the fat tissue layer A/B and the cell suspension C underneath it, a layer thickness-measuring unit 10 which measures the thickness of the fat tissue layer A/B based on the position of the boundary D detected by the image processing unit 9, a memory unit 11 which stores the cross-sectional area of the transparent container 3 and the volume of the fat tissue placed in the transparent container 3 before digestion, a fat volume-calculating unit 12 which calculates the volume of the fat tissue layer A/B by multiplying the thus measured thickness of the fat tissue layer A/B by the cross-sectional area of the transparent container 3, and a digestion termination-determining unit 13 which determines the termination of digestion based on the thus calculated volume of the fat tissue layer A/B and on the volume of the fat tissue before digestion that has been stored in the memory unit 11.

The image processing unit 9 detects the position of the boundary D between the fat tissue layer A/B and the cell suspension layer C and the position of the top surface E of the fat tissue layer A/B, for example, by binarizing the captured image based on the brightness value of each pixel. Based on the position of the top surface E of the fat tissue layer A/B and the position of the boundary D detected by the image processing unit 9, the layer thickness-measuring unit 10 calculates the thickness dimension of the fat tissue layer A/B located between these positions D and E.

Then, the fat volume-calculating unit 12 calculates the volume of the fat tissue layer A/B by multiplying the cross-sectional area of the transparent container 3 that has been previously stored in the memory unit 11 by the thickness dimension of the fat tissue layer calculated by the layer thickness-measuring unit.

The digestion termination-determining unit 13 determines the termination of digestion when the thus calculated volume of the fat tissue layer A/B decreases to a predetermined ratio with respect to the stored volume of the fat tissue at the time of digestion initiation.

In order to digest the fat tissue with use of the thus configured fat tissue digestion apparatus 2 according to this embodiment, a fat tissue collected from a human or another mammal, a protease such as trypsin, and a physiological saline are placed in the transparent container 3, and the swing mechanism 4 is operated.

Through the operation of the swing mechanism 4, the transparent container 3 is swung relative to the base 5, by which the fat tissue is stirred in the protease-containing physiological saline and is digested by the enzyme. As a result, with the passage of time, collagen which connects the fat tissue contained in the free lipid layer A is decomposed and thereby microstructures such as cells and extracellular tissue are lysed into the physiological saline, by which the layer A alters into the tissue layer B, and then into the cell suspension layer C as the digestion makes further progress.

The free lipid layer A and the tissue layer B get thinner as the stirring operation makes progress through the operation of the swing mechanism 4. In this process, the determination of digestion is performed through the operation of the fat digestion termination-determination apparatus 1. Specifically, the swinging operation of the transparent container 3 with the swing mechanism 4 is halted to leave the container still for a predetermined time, for example, 5 minutes. By so doing, the fat tissue layer A/B and the cell suspension layer C are separated.

Then, an image is captured by the camera 8 arranged to face the outer surface of the side wall of the transparent container 3, and the image processing unit 9 processes the captured image to thereby detect the boundary D between the fat tissue layer A/B and the cell suspension layer C. Thereafter, the thickness of the fat tissue layer A/B is measured based on the position of the boundary D through the operation of the layer thickness-measuring unit 10, and the volume of the fat tissue layer A/B is calculated through the operation of the fat volume-calculating unit 12. Then, when the volume of the fat tissue layer A/B decreases to a predetermined ratio with respect to the volume of the fat tissue layer at the time of digestion initiation, the termination of digestion is determined through the operation of the digestion termination-determining unit 13.

In this manner, according to the fat tissue digestion apparatus 2 of this embodiment, the decreased volume of the fat tissue can be accurately measured through the operation of the fat digestion termination-determination apparatus 1. That is to say, an advantage is given in which a group of fat-derived stem cells of constant quality can be obtained regardless of the individual difference in fat tissue, as compared to conventional methods in which the termination of fat tissue digestion is determined by counting the time.

In addition, since the termination of fat tissue digestion is determined based on the image captured by the camera 8 arranged to face the outer surface of the side wall of the transparent container 3, there is no need of taking out the fat tissue from the transparent container 3 and of measuring the weight and/or the volume thereof. Thus, the volume of the fat tissue can be readily calculated in a non-contact manner. As a result, another advantage is also given in which the termination of fat tissue digestion can be automatically determined.

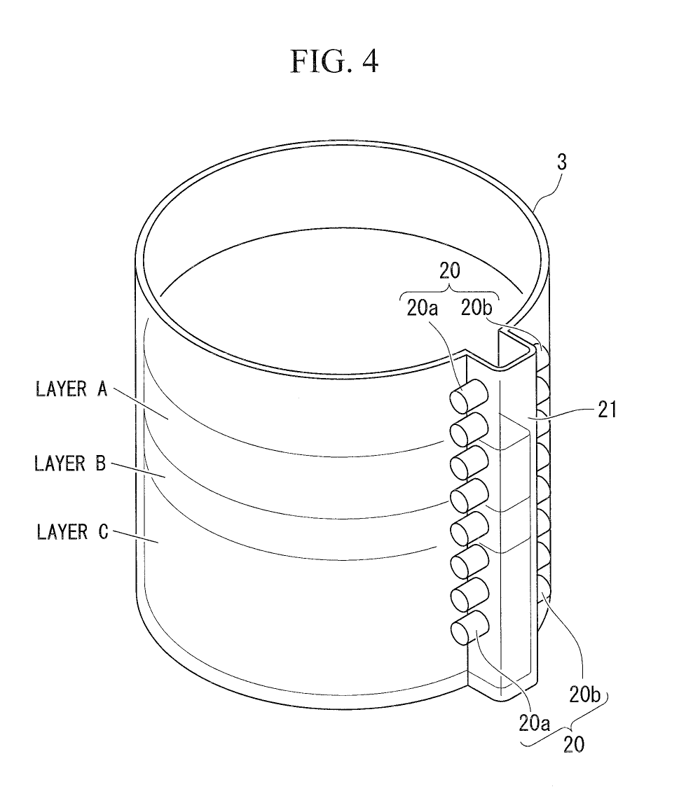

In this embodiment, the termination of fat tissue digestion is determined by processing the image captured by the camera 8 arranged to face the outer surface of the side wall of the transparent container 3; however, instead of this, infrared photoelectronic sensors 20 as shown in FIG. 4 may also be used. In this case, the arrangement may be such that a portion in the circumferential direction of the transparent container 3 is provided with a slender measurement projection 21 which is radially projected and vertically extended, and light emitting elements 20a and light receiving elements 20b are arranged to interpose the measurement projection 21 in the circumferential direction. The plurality of the infrared photoelectronic sensors 20 comprising the light emitting elements 20a and the light receiving elements 20b are adjacently arranged in the vertical direction.

With such a configuration, the position of the boundary D between the fat tissue layer A/B and the cell suspension C can be readily detected by utilizing the difference in the infrared absorbances of the fat tissue and the cell suspension.

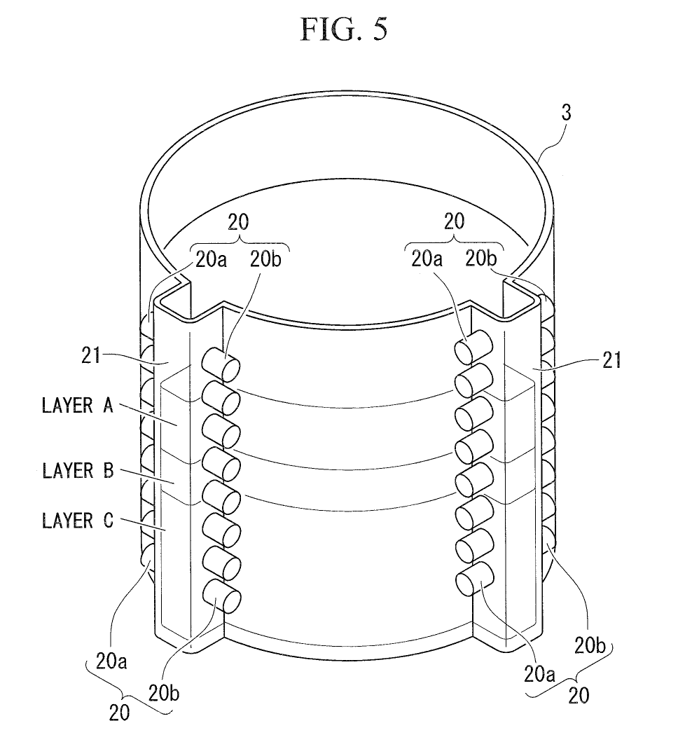

In addition, in this case, as shown in FIG. 5, further accurate detection is possible by improving the resolution for detecting the position of the boundary D by providing two or more measurement projections 21 at intervals in the circumferential direction and by shifting the height positions of the infrared photoelectronic sensors 20 for each measurement projection 21.

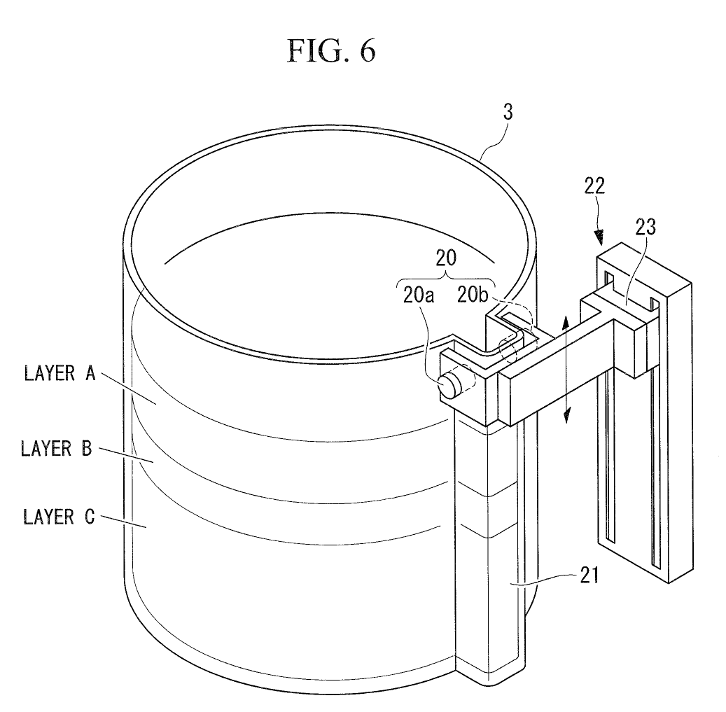

Moreover, instead of providing the plurality of the infrared photoelectronic sensors 20 in the vertical direction, the arrangement may also be such that, as shown in FIG. 6, a single infrared photoelectronic sensor 20 is vertically moved by an arbitrary lifting mechanism 22. Through the operation of the lifting mechanism 22 to operate the infrared photoelectronic sensor 20, it becomes possible to continuously search for the boundary D between the fat tissue layer A/B and the cell suspension layer C, so that the boundary position can be highly accurately detected. As for the lifting mechanism 22, any linear movement mechanism capable of vertically linear movement of a slider 23 may be employed.

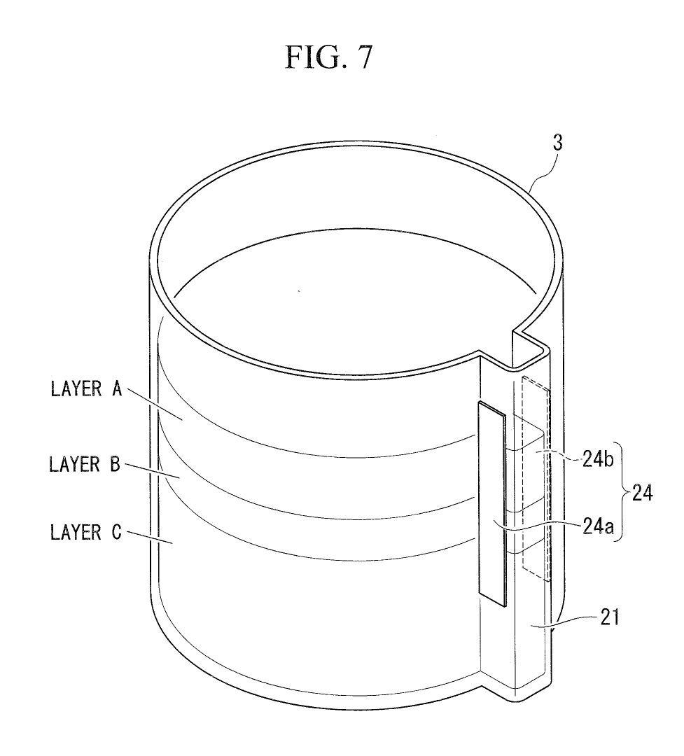

Furthermore, instead of the infrared photoelectronic sensor 20, there may also be arranged, as shown in FIG. 7, a capacitive sensor 24 comprising a pair of metal plates 24a and 24b which interpose the measurement projection 21 in the circumferential direction. By utilizing the phenomenon that the ratio and the capacitance relation between the fat tissue layer A/B and the cell suspension layer C interposed by the metal plates 24a and 24b of the capacitive sensor 24 are changed based on the difference in the conductivities of the fat tissue and of the cell suspension, the position of the boundary D formed therebetween can be accurately detected.

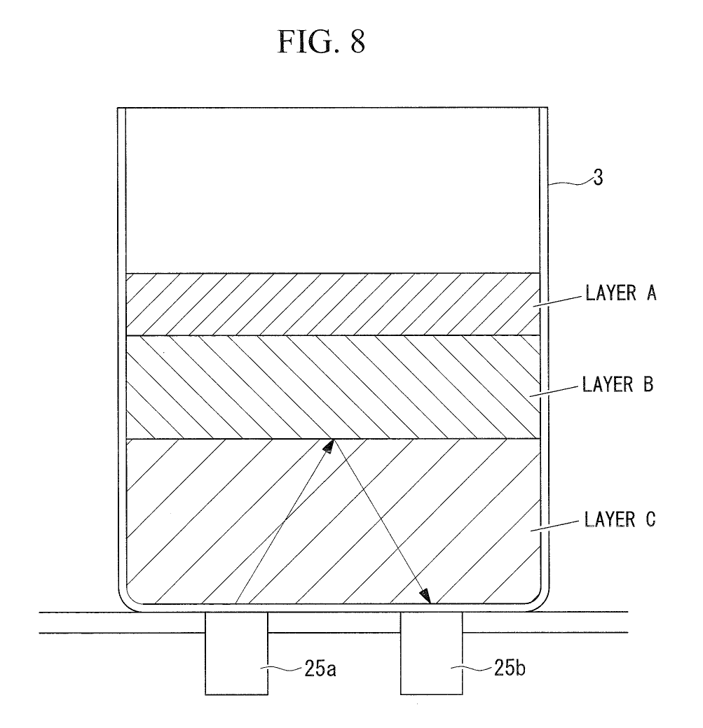

The arrangement may also be such that, as shown in FIG. 8, an ultrasonic generator 25a and an ultrasonic receiver 25b adhered to the bottom of the transparent container 3 are provided, and ultrasonic waves generated from the ultrasonic generator 25a and reflected by and returning from the surface of the boundary D between the fat tissue layer A/B and the cell suspension layer C are received by the ultrasonic receiver 25b, so as to thereby detect the position of the boundary D.

In addition, in this embodiment, the boundary D between the fat tissue layer A/B and the cell suspension layer C is obtained, and the change in the volume of the fat tissue layer A/B is monitored based on the boundary D to determine the termination of digestion. However, instead of this, the termination of digestion may also be determined at the time when the thickness dimension of the tissue layer B decreases to a predetermined dimension or lower value by utilizing the phenomenon that the tissue layer B is reduced with the passage of time.

In the abovementioned example, the volume of the fat tissue is measured and this data is stored in the memory unit in advance before the fat tissue is placed in the container. However, rather than measuring in advance, the fat volume may also be obtained by calculation based on the measured dimension of the fat tissue layer that floats upward in the physiological saline due to the relation of their specific gravities with use of the abovementioned configuration and apparatus before the addition of the enzyme. Furthermore, the amount of the enzyme necessary for digestion of this fat volume may be calculated based on this measured result by using the calculating function of the fat volume-calculating unit, and the necessary amount of the enzyme may be added based on this calculation result.

1. An apparatus for determining the termination of fat digestion for use in a fat tissue digestion apparatus which digests a fat tissue by placing and stirring the fat tissue with an enzyme solution, and any one of a physiological saline, a lactate Ringer's solution, and a buffer solution in a container, wherein the apparatus for determining the termination of fat digestion comprises:
a boundary-detecting unit which detects a boundary between a fat tissue layer formed in the container by being left still after stirring and a cell suspension layer located below the fat tissue layer, from outside the container;
a layer thickness-measuring unit which measures the thickness of the fat tissue layer based on the position of the boundary detected by the boundary-detecting unit;
a fat volume-calculating unit which calculates the volume of the fat tissue layer by multiplying the thus measured thickness of the fat tissue layer by the cross-sectional area of the container; and
a termination-determining unit which determines the termination of digestion based on the thus calculated volume of the fat tissue layer.

2. An apparatus for determining the termination of fat digestion according to claim 1, wherein
said container is transparent, and
said boundary-detecting unit comprises a camera which is arranged to face a side wall of said container for capturing an image of the fat tissue layer inside the container through the side wall of the container.

3. An apparatus for determining the termination of fat digestion according to claim 1, wherein
said container is transparent, and
said boundary-detecting unit comprises a light emitting element and a light receiving element which are opposingly arranged to interpose said container.

4. An apparatus for determining the termination of fat digestion according to claim 3, wherein a plurality of said light emitting elements and said light receiving elements are arranged in a vertical direction of the container.

5. An apparatus for determining the termination of fat digestion according to claim 3, comprising a movement mechanism which moves said light emitting element and said light receiving element in the vertical direction of the container.

6. A fat tissue digestion apparatus comprising the apparatus for determining the termination of fat digestion according to claim 1.
